(19) United States
(12) Patent Application Publication
(10) Pub. No.: US 2015/0011774 A1
(43) Pub. Date: Jan. 8, 2015

(54) PROCESS FOR THE PREPARATION OF AZILSARTAN MEDOXOMIL OR PHARMACEUTICALLY ACCEPTABLE SALTS THEREOF

(71) Applicant: RANBAXY LABORATORIES LIMITED, New Delhi, Delhi (IN)

(72) Inventors: Md Abul Kalam Azad, Karimgang (IN); Prakash Bhimaji Kshirsagar, Pune (IN); Shravan Kumar Singh, New Delhi (IN); Anand Prakash Tiwari, Sultanpur (IN); Kaptan Singh, Ghaziabad (IN); Mohan Prasad, Gurgaon (IN); Sudershan Kumar Arora, Gurgaon (IN)

(21) Appl. No.: 14/375,389

(22) PCT Filed: Jan. 30, 2013
(86) PCT No.: PCT/IB2013/050803

§ 371 Date: Jul. 29, 2014

(30) Foreign Application Priority Data
Feb. 2, 2012 (IN) 294/DEL/2012
Nov. 30, 2012 (IN) 3692/DEL/2012

(51) Int. Cl. C07D 413/14; C07D 235/26; C07D 405/12

(52) U.S. Cl. CPC C07D 413/14; C07D 405/12; C07D 235/26
USPC 548/132; 548/304.7

(57) ABSTRACT

The present invention relates to a process for the preparation of azilsartan medoxomil or pharmaceutically acceptable salts thereof.

### FIELD OF THE INVENTION

The present invention relates to a process for the preparation of azilsartan medoxomil or pharmaceutically acceptable salts thereof.

### BACKGROUND OF THE INVENTION

The drug substance used in the drug product formulation is the potassium salt of azilsartan medoxomil, also known by the United States accepted name of azilsartan kamedoxomil, is chemically described as (5-methyl-2-oxo-1,3-dioxol-4-yl)methyl 2-ethoxy-1-{[2′-(5-oxo-4,5-dihydro-1,2,4-oxadiazol-3-yl)biphenyl-4yl]methyl}-1H-benzimidazole-7-carboxylate monopotassium salt of Formula I.

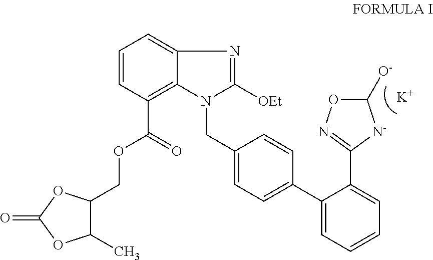 [SMILES: CCOc1nc2cccc(C(=O)OCC3OC(=O)OC3C)c2n1Cc1ccc(-c2ccccc2C2=NOC([O-])[N-]2)cc1 ; [K+]]

Azilsartan medoxomil of Formula II

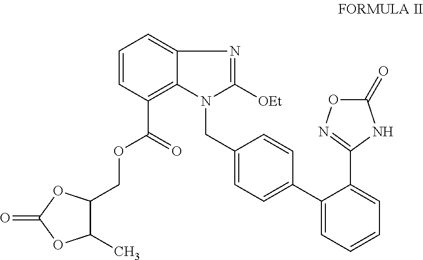 [SMILES: CCOc1nc2cccc(C(=O)OCC3OC(=O)OC3C)c2n1Cc1ccc(-c2ccccc2-c2noc(=O)[nH]2)cc1]

is a prodrug of azilsartan of Formula III

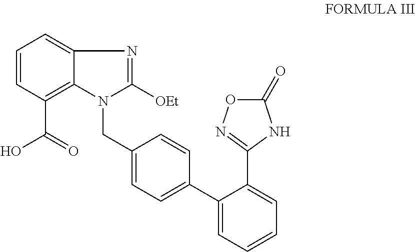 [SMILES: CCOc1nc2cccc(C(=O)O)c2n1Cc1ccc(-c2ccccc2-c2noc(=O)[nH]2)cc1]

which is a selective AT1 subtype angiotensin II receptor antagonist. Azilsartan medoxomil is an angiotensin II receptor blocker (ARB) indicated for the treatment of hypertension. It may be used alone or in combination with other antihypertensive agents.

Processes for the preparation of azilsartan medoxomil and its potassium salt are described in U.S. Pat. No. 7,157,584, and European Patent Nos. EP 1 718 641 and EP 2 119 715.

### SUMMARY OF THE INVENTION

The present invention relates to a process for the preparation of azilsartan medoxomil or its potassium salt.

### DETAILED DESCRIPTION OF THE INVENTION

A first aspect of the present invention provides a process for the preparation of potassium salt of azilsartan medoxomil which comprises:

a) reacting the compound of Formula IV or a salt thereof,

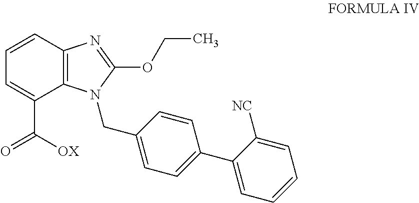

wherein X is hydrogen, the protecting group selected from alkyl, benzyl, substituted alkyl, or substituted aryl with hydroxyl amine, or its salt to form the compound of Formula V or a salt thereof,

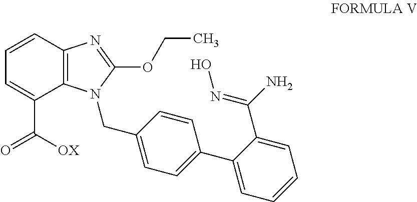

wherein X is hydrogen, the protecting group selected from alkyl, benzyl, substituted alkyl or substituted aryl;

b) optionally hydrolyzing the protecting group of the compound of Formula V;

c) reacting the compound of Formula V with the compound of Formula VI,

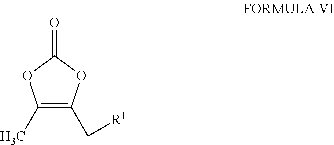

wherein R¹ is selected from halogen or hydroxyl atom,
to form the compound of Formula VII;

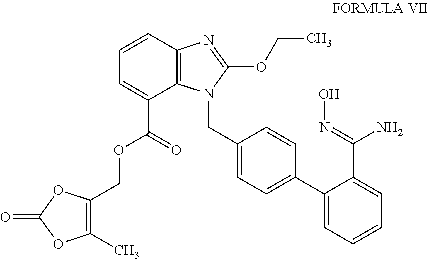 [SMILES: CCOc1nc2cccc(C(=O)OCc3oc(=O)oc3C)c2n1Cc1ccc(-c2ccccc2/C(N)=N/O)cc1]

d) optionally esterifying the compound of Formula VII to obtain the compound of Formula VIII,

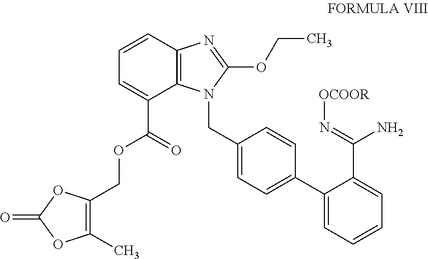

wherein R is hydrogen, alkyl, substituted alkyl, or an aryl group, wherein the aryl group may be further substituted by a nitro or chloro group;

e) cyclizing the compound of Formula VII or Formula VIII to form azilsartan medoxomil of Formula II;

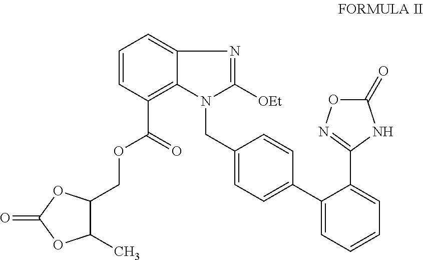 [SMILES: CCOc1nc2cccc(C(=O)OCC3OC(=O)OC3C)c2n1Cc1ccc(-c2ccccc2-c2noc(=O)[nH]2)cc1]

f) optionally isolating the azilsartan medoxomil of Formula II from the reaction mixture; and

g) converting azilsartan medoxomil to the potassium salt of azilsartan medoxomil of Formula I.

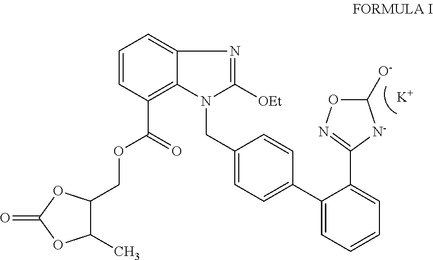 [SMILES: CCOc1nc2cccc(C(=O)OCC3OC(=O)OC3C)c2n1Cc1ccc(-c2ccccc2C2=NOC([O-])[N-]2)cc1 ; [K+]]

A second aspect of the present invention provides a process for the preparation of azilsartan medoxomil which comprises:

a) reacting the compound of Formula IV or a salt thereof,

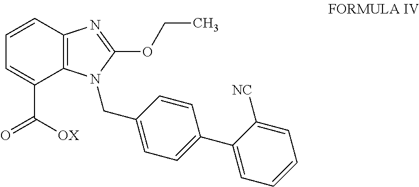

wherein X is hydrogen, the protecting group selected from alkyl, benzyl, substituted alkyl, or substituted aryl with hydroxyl amine or its salt, to form the compound of Formula V or a salt thereof,

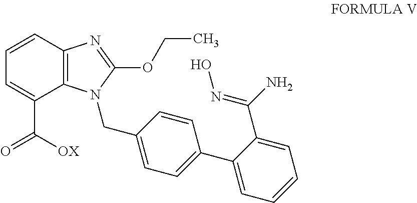

wherein X is hydrogen, the protecting group selected from alkyl, benzyl, substituted alkyl, or substituted aryl;

b) optionally hydrolyzing the protecting group of the compound of Formula V;

c) reacting the compound of Formula V with the compound of Formula VI

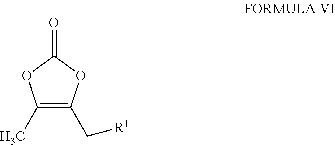

wherein R¹ is selected from halogen or hydroxyl atom
to form the compound of Formula VII;

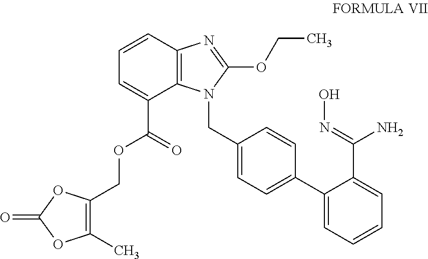 [SMILES: CCOc1nc2cccc(C(=O)OCc3oc(=O)oc3C)c2n1Cc1ccc(-c2ccccc2/C(N)=N/O)cc1]

d) optionally esterifying the compound of Formula VII to obtain the compound of Formula VIII,

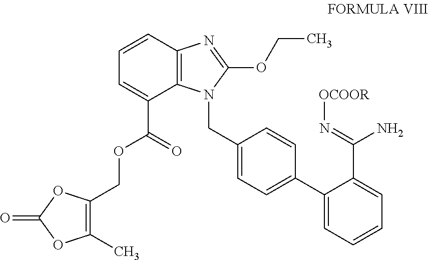

wherein R is hydrogen, alkyl, substituted alkyl, or aryl group wherein the aryl group may be further substituted by a nitro or chloro group;

e) cyclizing the compound of Formula VII or Formula VIII to form azilsartan medoxomil of Formula II; and

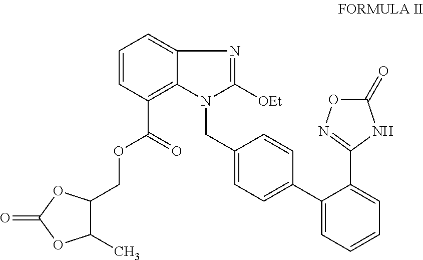 [SMILES: CCOc1nc2cccc(C(=O)OCC3OC(=O)OC3C)c2n1Cc1ccc(-c2ccccc2-c2noc(=O)[nH]2)cc1]

f) isolating the azilsartan medoxomil of Formula II from the reaction mixture.

A third aspect of the present invention provides a process for the preparation of the compound of Formula VII or Formula VIII which comprises:

a) reacting the compound of Formula IV or a salt thereof,

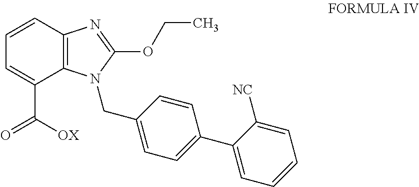

wherein X is hydrogen, the protecting group selected from alkyl, benzyl, substituted alkyl, or substituted aryl with hydroxyl amine or its salt to form the compound of Formula V or a salt thereof,

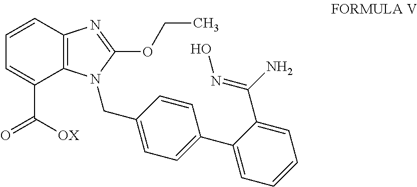

wherein X is hydrogen, the protecting group selected from alkyl, benzyl, substituted alkyl, or substituted aryl;

b) optionally hydrolyzing the protecting group of the compound of Formula V;

c) reacting the compound of Formula V with the compound of Formula VI,

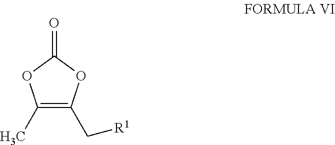

wherein R¹ is selected from halogen or hydroxyl atom
to form the compound of Formula VII;

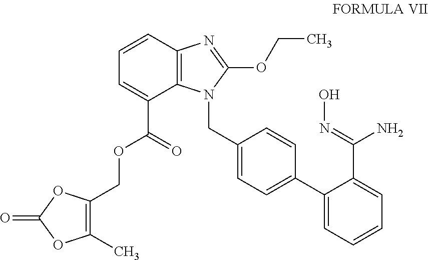 [SMILES: CCOc1nc2cccc(C(=O)OCc3oc(=O)oc3C)c2n1Cc1ccc(-c2ccccc2/C(N)=N/O)cc1]

d) optionally esterifying the compound of Formula VII to obtain the compound of Formula VIII,

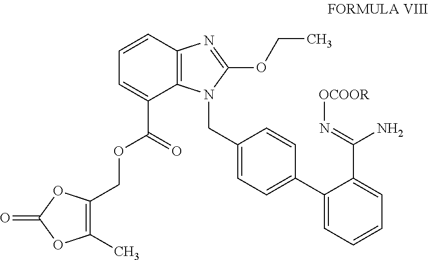

wherein R is hydrogen, alkyl, substituted alkyl, or aryl group, wherein the aryl group may be further substituted by a nitro or chloro group; and

e) isolating the compound of Formula VII or Formula VIII from the reaction mixture.

A fourth aspect of the present invention provides a process for the preparation of the compound of Formula IX, which comprises:

a) reacting the compound of Formula IV or a salt thereof,

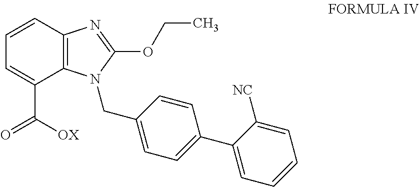

wherein X is hydrogen, the protecting group selected from alkyl, benzyl, substituted alkyl, or substituted aryl with hydroxyl amine or its salt to form the compound of Formula V or a salt thereof,

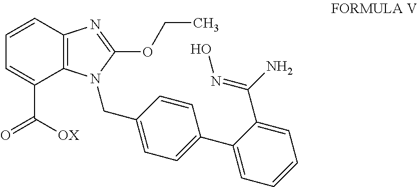

wherein X is hydrogen, the protecting group selected from alkyl, benzyl, substituted alkyl, or substituted aryl;

b) optionally hydrolyzing the protecting group of the compound of Formula V;

c) reacting the compound of Formula V with the compound of Formula VI

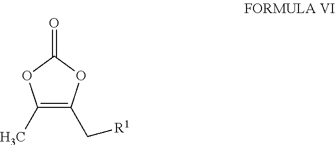

wherein R¹ is selected from halogen or hydroxyl atom
to form the compound of Formula VII;

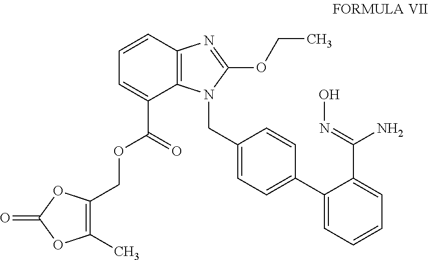 [SMILES: CCOc1nc2cccc(C(=O)OCc3oc(=O)oc3C)c2n1Cc1ccc(-c2ccccc2/C(N)=N/O)cc1]

d) esterifying the compound of Formula VII to obtain the compound of Formula IX; and

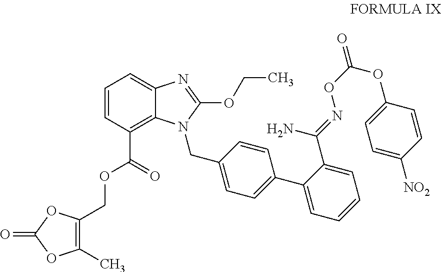 [SMILES: CCOc1nc2cccc(C(=O)OCc3oc(=O)oc3C)c2n1Cc1ccc(-c2ccccc2/C(N)=N/OC(=O)Oc2ccc([N+](=O)[O-])cc2)cc1]

e) isolating the compound of Formula IX from the reaction mixture thereof.

A fifth aspect of the present invention provides the compound of Formula VIII,

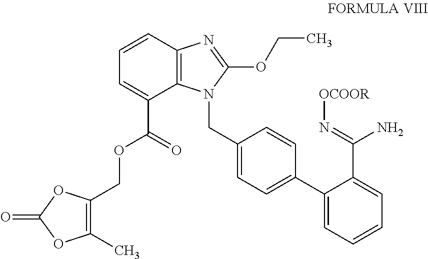

wherein R is hydrogen, alkyl, substituted alkyl, or an aryl group, wherein the aryl group may be further substituted by a nitro or chloro group.

A sixth aspect of the present invention provides the compound of Formula VII.

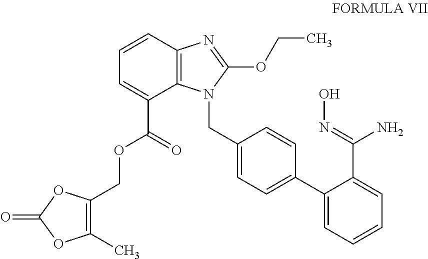 [SMILES: CCOc1nc2cccc(C(=O)OCc3oc(=O)oc3C)c2n1Cc1ccc(-c2ccccc2/C(N)=N/O)cc1]

A seventh aspect of the present invention provides the compound of Formula IX.

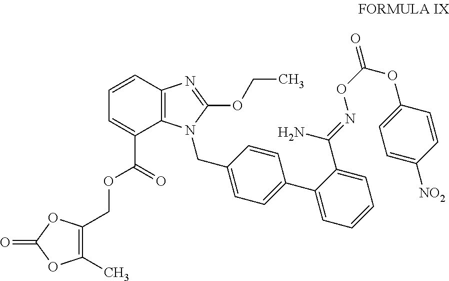 [SMILES: CCOc1nc2cccc(C(=O)OCc3oc(=O)oc3C)c2n1Cc1ccc(-c2ccccc2/C(N)=N/OC(=O)Oc2ccc([N+](=O)[O-])cc2)cc1]

An eighth aspect of the present invention provides the use of the compound of Formula VII for the preparation of azilsartan medoxomil or pharmaceutically acceptable salts thereof.

A ninth aspect of the present invention provides the use of the compound of Formula VIII for the preparation of azilsartan medoxomil or pharmaceutically acceptable salts thereof.

The compound of Formula IV may be reacted with hydroxyl amine in the presence of a base and solvent. Hydroxylamine may be used in the form of a salt, for example, hydrochloride salt. Examples of bases include hydroxide or alkoxide of alkali or alkaline earth metal or organic bases. Suitable alkoxides of alkali and alkaline earth metal include sodium methoxide or potassium methoxide. Suitable hydroxides of alkali and alkaline earth metals include sodium hydroxide or potassium hydroxide. Suitable organic bases include triethyl amine or tri-n-butyl amine. A preferable base may be sodium hydroxide.

The solvent may be selected from the group consisting of alcoholic solvents, polar solvents, or mixtures thereof. Suitable alcoholic solvents include methanol, ethanol, 1-butanol, or 2-butanol. Suitable polar solvents include dimethylformamide, dimethylacetamide, and dimethylsulphoxide. A preferable solvent includes methanol, dimethylacetamide, or mixtures thereof. Treatment of the compound of Formula IV with hydroxylamine may be carried out at 60° C. to 90° C., for example, at 70° C. to 80° C. Treatment may be carried out for 10 hours to 48 hours, for example, 24 hours.

The group X in the compound of Formula IV or Formula V may be selected from hydrogen, alkyl, benzyl, substituted alkyl, or substituted aryl. The alkyl group may be selected from methyl, ethyl, isopropyl, or a t-butyl group. A substituted aryl may be benzyl. The preferred group X in the compound of Formula IV or Formula V includes methyl. The protecting group in the compound of Formula V may optionally be hydrolyzed. The hydrolysis may be carried out using a base. A preferable base may be sodium hydroxide.

The salts of the compound of Formula IV or Formula V include sodium or potassium salt. The salts of the compound of Formula V may be formed in-situ and used further without isolating. The compound of Formula IV or Formula V may be converted to its salt by reacting with a suitable alkali metal hydroxide or an alkali metal carbonate. A suitable alkali metal hydroxide includes sodium hydroxide or potassium hydroxide. A suitable alkali metal carbonate includes sodium carbonate or potassium carbonate. The compound of Formula V or its salt may be isolated by filtration, distillation, decantation, vacuum drying, evaporation, or a combination thereof.

The compound of Formula V may be reacted with the compound of Formula VI in the presence of the solvent. The group R¹ in the compound of Formula VI may be selected from hydroxy or halogen atom. A suitable halogen atom includes chloro, bromo, or an iodo group. A preferable R¹ group includes a chloro atom. The solvent may be selected from the group consisting of polar solvents, halogenated hydrocarbon, or ketones. Suitable polar solvents include N,N-dimethylformamide, N,N-dimethylacetamide, or dimethylsulphoxide. Suitable halogenated hydrocarbon solvents include methylene chloride. Suitable ketonic solvents include acetone. A preferred solvent may be N,N-dimethylformamide.

The treatment of the compound of Formula V with the compound of Formula VI may be carried out at −10° C. to 60° C., for example, at 0° C. to 45° C. The treatment may be carried out for 20 hours to 30 hours, for example, 25 hours. The compound of Formula VII may be isolated by filtration, distillation, decantation, vacuum drying, evaporation, or a combination thereof.

The compound of Formula VII may be optionally esterified in the presence of a base and solvent. The R group in the compound of Formula VIII may be selected from a group consisting of hydrogen, alkyl, substituted alkyl, or an aryl group wherein the aryl group may be further substituted by a nitro or a chloro group. The R group may preferably be a 4-nitro phenyl group. Examples of a base include an organic or inorganic base. The inorganic base may be selected from hydroxide or carbonates of alkali or alkaline metal. The organic base may be selected from various amines, for example, triethyl amine. A suitable base includes triethyl amine or sodium bicarbonate.

The solvent may be selected from a group consisting of water, esters, halogenated hydrocarbon, ketone, ether, aromatic hydrocarbons, or mixtures thereof. The ester solvent may be, for example, ethyl acetate, isopropyl acetate, butyl acetate, isobutyl acetate, or a mixture thereof. The halogenated hydrocarbon may be, for example, dichloromethane. The ketone solvent may be, for example, acetone. The ether solvent may be, for example, tetrahydrofuran. The aromatic hydrocarbon solvent may be, for example, toluene. The solvent may preferably be acetone, isobutyl acetate, or toluene. The esterifying agent may be selected from alkyl or aryl chloroformate, both substituted and unsubstituted. The alkyl chloroformate may be, for example, ethyl chloroformate. The aryl chloroformate may be, for example, phenyl chloroformate, 4-nitro phenyl chloroformate, or 4-chlorophenyl chloroformate. The esterifying agent may preferably be 4-nitrophenyl chloroformate. The esterification of the compound of Formula VII may be carried out at −10° C. to 50° C., for example, at 0° C. to 35° C. The treatment may be carried out for 2 hours to 4 hours, for example, 3 hours. The compound of Formula VIII may be isolated by filtration, distillation, decantation, vacuum drying, evaporation, or a combination thereof.

The compound of Formula VII or Formula VIII may be cyclized in the presence of a solvent. The compound of Formula VII or Formula VIII may be used in the cyclization step in the solution form without isolating. The cyclization may be a thermal or chemical induced cyclization. Chemical induced cyclization may be carried out using a cyclizing agent. A suitable cyclizing agent may include carbodiimidazole, phosgene, or triphosgene.

The solvent may be selected from the group consisting of water, ketone, halogenated hydrocarbon, alcohols, ether, esters, or mixtures thereof. An ester solvent may be, for example, ethyl acetate, isopropyl acetate, butyl acetate, isobutyl acetate, or a mixture thereof. A halogenated hydrocarbon may be, for example, dichloromethane. An ether solvent may be, for example, tetrahydrofuran. A ketone solvent may be, for example, acetone. An aromatic hydrocarbon solvent may be, for example, toluene. Examples of alcoholic solvents include ethanol, isopropanol, or isobutanol. The solvent may preferably be acetone, isobutyl acetate, or toluene. Cyclization of the compound of Formula VII or Formula VIII may be carried out at 10° C. to 125° C., for example, at 20° C. to 115° C. The treatment may be carried out for 10 hours to 15 hours, for example, 13 hours. The compound of azilsartan medoxomil of Formula II may be isolated by filtration, distillation, decantation, vacuum drying, evaporation, or a combination thereof.

The compound of azilsartan medoxomil of Formula II may optionally be purified using various solvents. The solvent used for purification of the compound of azilsartan medoxomil of Formula II may be selected from the group consisting of ketone, halogenated hydrocarbon, alcohols, esters, or mixtures thereof. The ester solvent may be, for example, ethyl acetate, isopropyl acetate, butyl acetate, isobutyl acetate, or a mixture thereof. The halogenated hydrocarbon may be, for example, dichloromethane. The ketone solvent may be, for example, acetone. The aromatic hydrocarbon solvent may be, for example, toluene. Examples of alcoholic solvents include ethanol, isopropanol, or isobutanol. The solvent may preferably be acetone.

Azilsartan medoxomil may optionally be converted to potassium salt of azilsartan medoxomil by reacting with a potassium source in the presence of a solvent. The suitable potassium source may include potassium-2-ethylhexanoate. The solvent may be selected from the group consisting of ketone, aromatic hydrocarbon, or mixtures thereof. An example of aromatic hydrocarbon solvents includes toluene. An example of ketone solvents includes acetone, methyl isobutyl ketone, methyl ethyl ketone, or methyl isopropyl ketone. Preferable solvents include acetone. The compound of potassium salt of azilsartan medoxomil of Formula I may be isolated by filtration, distillation, decantation, vacuum drying, evaporation, or a combination thereof.

The compound of potassium salt of azilsartan medoxomil of Formula I, the compound of azilsartan medoxomil of Formula II, the compound of Formula VII, and the compound of Formula VIII may be further characterized by X-ray Powder Diffraction Pattern (XRPD) pattern.

While the present invention has been described in terms of its specific embodiments, certain modifications and equivalents will be apparent to those skilled in the art and are intended to be included within the scope of the present invention.

### EXAMPLES

### Examples 1A and 1B

### Example 1A

### Preparation of Methyl 2-Ethoxy-1-{[2′-(N′-Hydroxy Carb Amimidoyl)Biphenyl-4-Yl]Methyl}-1H-Benzimidazole-7-Carboxylate (Formula V)

Methanol (400 mL) and sodium hydroxide (56.44 g) were added under a nitrogen atmosphere and stirred at 20° C. to 30° C. to get a clear solution. Dimethylacetamide (750 mL) and hydroxylamine hydrochloride (101.45 g) were added under a nitrogen atmosphere and stirred at 20° C. to 30° C. to get clear solution. Methanolic sodium hydroxide solution was added to the solution of hydroxylamine hydrochloride in dimethylacetamide at 25° C. to 30° C. The reaction mixture was stirred for 30 minutes. Methyl-1-[(2′-cyanobiphenyl-4-yl)methyl]-2-ethoxy-1H-benzimidazole-7-carboxylate (100 g) was added to the reaction mixture at 25° C. to 30° C. and heated to 70° C. to 75° C. for 16 hours to 20 hours. The reaction mixture was cooled to 10° C. to 15° C. The reaction mixture was added to deionized water (1000 mL) at 10° C. to 25° C. The pH of the reaction mixture was adjusted to 0.8 to 1.2 using concentrated hydrochloric acid (150 mL). The reaction mixture was stirred for 30 minutes at 20° C. to 30° C. The reaction mixture was filtered through celite and washed with deionized water (100 mL). The aqueous layer was washed with toluene (500 mL) at 25° C. to 30° C. and the pH of the aqueous layer was adjusted to 8.8 to 9.2 using 30% solution of sodium carbonate (500 mL) at 20° C. to 30° C. The reaction mixture was stirred for 3 hours to 4 hours at 25° C. to 30° C. The reaction mixture was filtered and washed with deionized water (100 mL) at 20° C. to 30° C. Isobutanol (500 mL) was added to the reaction mixture at 20° C. to 30° C. and the reaction mixture was heated to 90° C. to 95° C. The reaction mixture was stirred for 2 hours at 90° C. to 95° C., cooled to 25° C. to 30° C., and stirred for 4 to 6 hours at 25° C. to 30° C. The reaction mixture was filtered and washed with isobutanol (100 mL) at 20° C. to 30° C. The reaction mixture was dried under vacuum for 30 minutes at 20° C. to 30° C. and then at 45° C. to 50° C. to obtain the title compound.

Yield: 55 g

### Example 1B

### Preparation of Methyl 2-Ethoxy-1-{[2′-(N′-Hydroxy Carb Amimidoyl) Biphenyl-4-Yl]Methyl}-1H-Benzimidazole-7-Carboxylate (Formula V)

Dimethyl sulfoxide (75 mL) and hydroxylamine hydrochloride (6 g) were stirred at 20° C. to 30° C. Sodium bicarbonate (9 g) was added to the solution and stirred at 45° C. to 50° C. for 1 hour. Methyl-1-[(2′-cyanobiphenyl-4-yl)methyl]-2-ethoxy-1H-benzimidazole-7-carboxylate (3 g) was added to the reaction mixture and heated at 80° C. to 85° C. for 20 hours. The reaction mixture was cooled to 20° C. to 30° C. and de-ionized water (75 mL) was added to the reaction mixture. The solid obtained was filtered and washed with water (75 mL). The solid obtained was purified in 2-butanol (15 mL) at 90° C. to 95° C. for 4 hours and further cooled to 20° C. to 30° C. for 4 hours. The solid obtained was filtered, washed with 2-butanol (6 mL), and dried to obtain the title compound.

Yield: 2.75 g (85%)

HPLC Purity: 96.89%

### Examples 2A and 2B

### Example 2A

### Preparation of 2-Ethoxy-1-{[2′-(N′-Hydroxy Carb Amimidoyl)Biphenyl-4-Yl]Methyl}-1H-Benzimidazole-7-Carboxylic Acid (Formula V)

Methyl 2-ethoxy-1-{[2′-(N′-hydroxycarbamimidoyl)biphenyl-4-yl]methyl}-1H-benzimidazole-7-carboxylate (10 g) prepared in Example 1A, deionized water (180 mL), methanol (50 mL) and tetrahydrofuran (50 mL) were added to a round-bottom flask and stirred at 20° C. to 30° C. An aqueous sodium hydroxide solution (1 g in 20 mL deionized water) was added to the reaction mixture at 20° C. to 30° C. The temperature of the reaction mixture was increased to 45° C. to 50° C. and the reaction mixture was stirred for 8 to 10 hours. Tetrahydrofuran and the methanol mixture were recovered completely under vacuum. The reaction mixture was washed with toluene (100 mL) at 20° C. to 30° C. The pH of the aqueous reaction mixture was adjusted to 4.0 to 4.5 using concentrated hydrochloric acid. The reaction mixture was filtered and washed with deionized water (2×40 mL). The reaction mixture was dried at 50° C. to obtain the title compound.

Yield: 8.1 g

(M+H)⁺: m/z=431.2.

¹H NMR (400 MHz, DMSO-d₆): δ 1.39-1.43 (t, 3H), 4.58-4.63 (q, 2H), 5.58 (s, 2H), 5.64 (s, 2H), 6.98-7.67 (m, 11H).

### Example 2B

### Preparation of 2-Ethoxy-1-{[2′-(N′-Hydroxy Carb Amimidoyl)Biphenyl-4-Yl]Methyl}-1H-Benzimidazole-7-Carboxylic Acid (Formula V)

The methyl 2-ethoxy-1-{[2′-(N′-hydroxycarbamimidoyl)biphenyl-4-yl]methyl}-1H-benzimidazole-7-carboxylate (60 g) prepared in Example 1B and a solution of sodium hydroxide (8.1 g) in de-ionized water (120 mL) were added to tetrahydrofuran (240 mL) and heated at 60° C. to 65° C. for 8 hours. The reaction mixture was cooled to 20° C. to 30° C. and de-ionized water (120 ml) was added to the reaction mixture. 2N Hydrochloric acid (96 ml) was added to the reaction mixture to adjust the pH to 6.0 to 6.2. The solid obtained was filtered at 20° C. to 30° C. and washed with a solution of tetrahydrofuran (60 mL) and de-ionized water (60 mL). The solid material obtained was dried at 40° C. to obtain the title compound.

Yield: 52.0 g

HPLC purity: 98.64%

### Example 3

### Preparation of Sodium Salt of 2-Ethoxy-1-{[2′-(N′-Hydroxy Carb Amimidoyl)Biphenyl-4-Yl]Methyl}-1H-Benzimidazole-7-Carboxylic Acid (Formula V Sodium Salt)

The 2-Ethoxy-1-{[2′-(N′-hydroxycarbamimidoyl)biphenyl-4-yl]methyl}-1H-benzimidazole-7-carboxylic acid (25 g) prepared in Example 2A and methanol (125 mL) were added to a round-bottom flask at 20° C. to 30° C. A solution of sodium hydroxide was prepared by dissolving sodium hydroxide (2.33 g) in methanol (62.5 mL) at 20° C. to 30° C. This solution was added to the round-bottom flask at 20° C. to 30° C. and stirred for 1 hour. Methanol was recovered from the reaction mixture. Acetone (250 mL) was added to the reaction mixture and stirred for 2 hours at 20° C. to 30° C. The reaction mixture was filtered and washed with acetone (50 mL). The reaction mixture was dried under vacuum at 40° C. to obtain the title compound.

Yield: 26.0 g

### Example 4

### Preparation of (5-Methyl-2-Oxo-1,3-Dioxol-4-Yl)Methyl 2-Ethoxy-1-{[2′-(N′-Hydroxycarbamimidoyl)Biphenyl-4-Yl]-Methyl}-1H-Benzimidazole-7-Carboxylate (Formula VII)

The sodium salt of 2-ethoxy-1-{[2′-(N′-hydroxycarbamimidoyl)-biphenyl-4-yl]-methyl}-1H-benzimidazole-7-carboxylic acid (1g) prepared in Example 3 and N,N-dimethylformamide (25 mL) were combined and stirred at 20° C. to 30° C. The reaction mixture was cooled to 0° C. to 5° C. A solution of 4-(chloromethyl)-5-methyl-1,3-dioxol-2-one (0.36 g) in N,N-dimethylformamide (10 mL) was added to the reaction mixture at 0° C. to 5° C. and stirred for 30 minutes. The temperature of the reaction mixture was raised to 20° C. to 30° C. and stirred for 20 hours. Deionized water (125 mL) was added to the reaction mixture at 15° C. to 25° C. and stirred for 3 hours at 20° C. to 30° C. The reaction mixture was filtered, washed with deionized water (25 mL), and dried at 45° C. under vacuum to obtain the title compound.

Yield: 0.9 g.

(M+H)⁺: m/z=543.1.

¹H NMR (400 MHz, CDCl₃): δ 1.47-1.5 (t, 3H), 2.15 (s, 3H), 4.64-4.69 (q, 2H), 4.9 (s, 2H), 5.6 (s, 2H), 6.9-7.58 (m, 11H).

### Examples 5A and 5B

### Example 5A

### Preparation of (5-Methyl-2-Oxo-1,3-Dioxol-4-Yl)Methyl 2-Ethoxy-1-{[2′-(N′-Hydroxycarbamimidoyl)Biphenyl-4-Yl]-Methyl}-1H-Benzimidazole-7-Carboxylate (Formula VII)

The 2-ethoxy-1-{[2′-(N′-hydroxycarbamimidoyl)biphenyl-4-yl]methyl}-1H-benzimidazole-7-carboxylic acid (100 g) prepared in Example 2A, dimethyl acetamide (450 mL), and potassium carbonate (20.9 g) were heated to 80° C. to 85° C. for 2 hours. The reaction mixture was cooled to 45° C. to 50° C. A solution of 4-(chloromethyl)-5-methyl-1,3-dioxol-2-one (41.44 g) in N,N-dimethylformamide (50 mL) was added to the reaction mixture at 40° C. to 45° C. The reaction mixture was stirred at 45° C. to 55° C. for 6 to 8 hours. The reaction mixture was cooled to 15° C. to 20° C. A solution of sodium bicarbonate (100 g) in water (2000 mL) was added to the reaction mixture. The solid obtained was filtered and washed with water (500 mL) and dried below 50° C. to obtain the title compound.

Yield: 90 g

### Example 5B

### Preparation of (5-Methyl-2-Oxo-1,3-Dioxol-4-Yl)Methyl 2-Ethoxy-1-{[2′-(N′-Hydroxycarbamimidoyl)Biphenyl-4-Yl]-Methyl}-1H-Benzimidazole-7-Carboxylate (Formula VII)

The 2-ethoxy-1-{[2′-(N-hydroxycarbamimidoyl)biphenyl-4-yl]methyl}-1H-benzimidazole-7-carboxylic acid (25 g) prepared in Example 2B, acetone (250 mL), and potassium carbonate (8.1 g) were heated to 45° C. to 50° C. for 2 hours. The reaction mixture was cooled to 25° C. to 30° C. Tetrabutyl ammonium bromide (0.94 g), sodium iodide (0.44 g), and 4-(chloromethyl)-5-methyl-1,3-dioxol-2-one (9.5 g) were added to the reaction mixture and stirred for 4 hours at 25° C. to 30° C. The reaction mixture was cooled to 5° C. to 10° C. 2N Hydrochloric acid (50 mL) was added to the reaction mixture to adjust the pH to 1.5 to 2.0. De-ionized water (250 mL) was added to the reaction mixture and washed with toluene (50 mL). Sodium bicarbonate solution (12.5 g sodium bicarbonate in 150 mL de-ionized water) was added to the reaction mixture to adjust the pH to 7.0 to 7.5. The solid obtained was filtered, washed with de-ionized water (50 mL) and dried. The reaction mixture was purified with ethyl acetate (150 mL) to obtain the title compound.

Yield: 22.5 g

HPLC purity: 96.78%

### Example 6

### Preparation of (5-Methyl-2-Oxo-1,3-Dioxol-4-Yl)Methyl 2-Ethoxy-1-[(2′-{N′-[(Ethoxycarbonyl)Oxy]Carbamimidoyl}-Biphenyl-4-Yl)-Methyl]-1H-Benzimidazole-7-Carboxylate (Formula VIII)

Dichloromethane (100 mL), (5-methyl-2-oxo-1,3-dioxol-4-yl)methyl 2-ethoxy-1-{[2′-(N′-hydroxycarbamimidoyl)biphenyl-4-yl]methyl}-1H-benzimidazole-7-carboxylate (6.5 g) prepared in Example 5A, and triethylamine (1.45 g) were added to a round bottom flask at 20° C. to 30° C. The reaction mixture was cooled to 0° C. to 5° C. A solution of ethyl chloroformate (1.43 g) in dichloromethane (30 mL) was added to the reaction mixture at 0° C. to 5° C. and stirred for 30 minutes. The temperature of the reaction mixture was raised to 20° C. to 30° C. and stirred for 1 hour. A solution of sodium bicarbonate (4.88 g) in de-ionized water (100 mL) was added to the reaction mixture at 20° C. to 30° C. and stirred for 20 minutes. The organic layer was separated and washed with a solution of sodium chloride (45 g) in de-ionized water (150 mL) at 20° C. to 30° C. The organic layer was separated and concentrated under vacuum at 35° C. to 40° C. The reaction mixture was filtered, washed with diisopropyl ether (15 mL), and dried at 30° C. to 35° C. under vacuum to obtain the title compound.

Yield: 6.8 g

(M+H)⁺: m/z=615.4.

¹H NMR (400 MHz, CDCl₃): δ 1.32-1.36 (t, 3H), 1.46-1.5 (t, 3H), 2.17 (s, 3H), 4.27-4.32 (q, 2H), 4.64-4.69 (q, 2H), 4.93 (s, 2H), 5.64 (s, 2H), 6.95-7.78 (m, 11H).

### Example 7

### Preparation of (5-Methyl-2-Oxo-1,3-Dioxol-4-Yl)Methyl 2-Ethoxy-1-{[2′-(N′-{[(4-Nitrophenoxy)Carbonyl]oxy}Carbamimidoyl)Biphenyl-4-Yl]Methyl}-1H-Benzimidazole-7-Carboxylate (Formula IX)

De-ionized water (125 mL) and sodium bicarbonate (12.5 g) were stirred at 20° C. to 30° C. Dichloromethane (175 mL) was added to the reaction mixture. The (5-methyl-2-oxo-1,3-dioxol-4-yl)methyl 2-ethoxy-1-{[2′-(n′-hydroxycarbamimidoyl)biphenyl-4-yl]-methyl}-1H-benzimidazole-7-carboxylate (25 g) prepared in Example 5B was added to the reaction mixture. A solution of 4-nitrophenyl chloroformate (9.3 g) in dichloromethane (75 mL) was added to the reaction mixture at 20° C. to 30° C. and stirred for 1 hour. The reaction mixture obtained was allowed to settle and the layers were separated. The organic layer was washed with de-ionized water (125 mL). Dichloromethane was recovered to obtain the title compound.

Yield: 32.0 g (98%).

(M+H)⁺: m/z=708.2.

¹H NMR (400 MHz, CDCl₃): δ 1.45-1.49 (t, 3H), 2.16 (s, 3H), 4.63-4.68 (q, 2H), 4.94 (s, 2H), 5.65 (s, 2H), 6.95-8.28 (m, 15H).

### Examples 8A, 8B, 8C, 8D, and 8E

### Example 8A

### Preparation of (5-Methyl-2-Oxo-1,3-Dioxol-4-Yl)Methyl 2-Ethoxy-1-{[2′-(5-Oxo-4,5-Dihydro-1,2,4-Oxadiazol-3-Yl)Biphenyl-4-Yl]Methyl}-1H-Benzimidazole-7-Carboxylate (Formula II)

The (5-methyl-2-oxo-1,3-dioxol-4-yl)methyl-2-ethoxy-1-[(2′-{N′-[(ethoxycarbonyl)-oxy]-carbamimidoyl}-biphenyl-4-yl)-methyl]-1H-benzimidazole-7-carboxylate (6 g) prepared in Example 6 and methyl isobutyl ketone (90 mL) were added to a round-bottom flask at 20° C. to 30° C. The temperature of the reaction mixture was raised to 110° C. to 115° C. and stirred for 10 hours at 110° C. to 115° C. The reaction mixture was cooled to 20° C. to 30° C. The solvent was recovered at 50° C. under vacuum. Dichloromethane (240 mL) was added to the reaction mixture and stirred for 30 minutes. The reaction mixture was washed with an aqueous 0.5 N hydrochloric acid solution for 15 minutes, followed by washing with aqueous sodium bicarbonate solution (120 mL), and finally washed with an aqueous sodium chloride solution (120 mL). Dichloromethane was recovered at 40° C. under vacuum. The solid obtained was crystallized using acetone (60 mL) to obtain the title compound.

Yield: 2.7 g

HPLC Purity: 99.14%

### Example 8B

### Preparation of (5-Methyl-2-Oxo-1,3-Dioxol-4-Yl)Methyl 2-Ethoxy-1-{[2′-(5-Oxo-4,5-Dihydro-1,2,4-Oxadiazol-3-Yl)Biphenyl-4-Yl]Methyl}-1H-Benzimidazole-7-Carboxylate (Formula II)

The 5-methyl-2-oxo-1,3-dioxol-4-yl-methyl 2-ethoxy-1-{[2′-(n′-{[(4-nitrophenoxy)carbonyl]oxy}carbamimidoyl)biphenyl-4-yl]methyl}-1H-benzimidazole-7-carboxylate (32.0 g) prepared in Example 7 was added to isobutyl acetate (250 mL) at 20° C. to 30° C. The temperature of the reaction mixture was raised to 55° C. to 60° C. and stirred for 12 hours. The reaction mixture was cooled to 20° C. to 30° C. and filtered. The reaction mixture was washed with isobutyl acetate (50 ml) at 20° C. to 30° C. The reaction mixture obtained was dried under vacuum at 40° C. to obtain the title compound.

Yield: 23.0 g (87%)

HPLC Purity: 99.15%

### Example 8C

### Preparation of (5-Methyl-2-Oxo-1,3-Dioxol-4-Yl)Methyl 2-Ethoxy-1-{[2′-(5-Oxo-4,5-Dihydro-1,2,4-Oxadiazol-3-Yl)Biphenyl-4-Yl]Methyl}-1H-Benzimidazole-7-Carboxylate (Formula II)

The 5-methyl-2-oxo-1,3-dioxol-4-yl-methyl 2-ethoxy-1-{[2′-(n′-{[(4-nitrophenoxy)carbonyl]oxy}carbamimidoyl)biphenyl-4-yl]methyl}-1H-benzimidazole-7-carboxylate (32.0 g) prepared in Example 7 was added to acetone (250 mL) at 20° C. to 30° C. The temperature of the reaction mixture was raised to 55° C. to 60° C. and stirred for 12 hours. The reaction mixture was cooled to 0° C. to 5° C. and stirred for 4 hours. The reaction mixture was filtered and washed with acetone (25 ml) at 20° C. to 30° C. The reaction mixture obtained was dried under vacuum at 40° C. to obtain the title compound.

Yield: 21.0 g (79%)

HPLC Purity: 98.83%

### Example 8D

### Preparation of (5-Methyl-2-Oxo-1,3-Dioxol-4-Yl)Methyl 2-Ethoxy-1-{[2′-(5-Oxo-4,5-Dihydro-1,2,4-Oxadiazol-3-Yl)Biphenyl-4-Yl]Methyl}-1H-Benzimidazole-7-Carboxylate (Formula II)

The 5-methyl-2-oxo-1,3-dioxol-4-yl-methyl 2-ethoxy-1-{[2′-(n′-{[(4-nitrophenoxy)carbonyl]oxy}carbamimidoyl)biphenyl-4-yl]methyl}-1H-benzimidazole-7-carboxylate (6.39 g) prepared in Example 7 was added to toluene (50 mL) at 20° C. to 30° C. The temperature of the reaction mixture was raised to 70° C. and stirred for 12 hours. The reaction mixture was cooled to 20° C. to 30° C. and filtered. The reaction mixture was washed with toluene (50 ml) at 20° C. to 30° C. The reaction mixture obtained was dried under vacuum at 40° C. to obtain the title compound.

Yield: 4.63 g (87%)

HPLC Purity: 97.15%

### Example 8E

### Preparation of (5-Methyl-2-Oxo-1,3-Dioxol-4-Yl)Methyl 2-Ethoxy-1-{[2′-(5-Oxo-4,5-Dihydro-1,2,4-Oxadiazol-3-Yl)Biphenyl-4-Yl]Methyl}-1H-Benzimidazole-7-Carboxylate (Formula II)

Isobutyl acetate (250 mL) was added to 5-methyl-2-oxo-1,3-dioxol-4-yl-methyl 2-ethoxy-1-{[2′-(n′-{[(4-nitrophenoxy)carbonyl]oxy}carbamimidoyl)biphenyl-4-yl]methyl}-1H-benzimidazole-7-carboxylate (25 g) prepared in Example 7 at 20° C. to 30° C. The temperature was increased to 65° C. to 70° C. and stirred for 12 hours. The reaction mixture was cooled to 20° C. to 30° C. and stirred for 4 hours. The solid obtained was filtered and washed with isobutyl acetate (50 mL). The reaction mixture was dissolved in a solution of dichloromethane (400 mL), acetone (60 mL), and saturated sodium bicarbonate solution (250 mL) at 30° C. to 35° C. and stirred for 1 hour. The layers obtained were separated and the dichloromethane layer was washed with de-ionized water (250 mL) at 20° C. to 30° C. The layers obtained were separated and the dichloromethane layer was recovered under vacuum at 25° C. to 35° C. The reaction mixture was dissolved in acetone (250 mL) at 55° C. to 56° C. and cooled to 0° C. to 5° C. for 4 hours. The residue obtained was filtered and washed with acetone (25 mL). The residue obtained was dried at 40° C. under vacuum to obtain the title compound.

Yield: 16 g

HPLC Purity: 99.91%

### Example 9

### Preparation of (5-Methyl-2-Oxo-1,3-Dioxol-4-Yl)Methyl 2-Ethoxy-1-{[2′-(5-Oxo-4,5-Dihydro-1,2,4-Oxadiazol-3 -Yl)Biphenyl-4-Yl]Methyl}-1H-Benzimidazole-7-Carboxylate (Formula II)

De-ionized water (125 mL) was added to sodium bicarbonate (12.5 g) at 20° C. to 30° C. and stirred. The (5-methyl-2-oxo-1,3-dioxol-4-yl)methyl 2-ethoxy-1-{[2′-(n′-hydroxycarbamimidoyl)biphenyl-4-yl]-methyl}-1H-benzimidazole-7-carboxylate (25 g) prepared in Example 5B and dichloromethane (175 mL) were added to the reaction mixture at 20° C. to 30° C. A solution of 4-nitrophenyl chloroformate (9.3 g) in dichloromethane (75 mL) was added to the reaction mixture at 20° C. to 30° C. and stirred for 1 hour. The layers obtained were separated and the organic layer was washed with de-ionized water (125 mL). Dichloromethane was completely recovered to obtain the solid material. Acetone (125 mL) was added to the reaction mixture and the temperature was increased to 55° C. to 57° C. The reaction mixture was stirred for 12 hours, cooled to 20° C. to 25° C., and stirred for 4 hours. The reaction mixture was filtered and washed with acetone (25 mL). The reaction mixture was dissolved in dichloromethane (500 mL), acetone (75 mL), and a solution of sodium bicarbonate (250 mL) at 35° C. to 40° C. and stirred for 1 hour. The layers obtained were separated and the dichloromethane layer was washed with de-ionized water (250 mL) at 20° C. to 30° C. The layers obtained were separated and dichloromethane was recovered under vacuum at 25° C. to 35° C. The solid material obtained was slurried in acetone (75 mL) at 20° C. to 25° C. for 4 hours. The solid obtained was filtered and washed with acetone (25 mL). The solid obtained was dried under vacuum at 40° C. to obtain the title compound.

Yield: 20 g

HPLC Purity: 99.92%.

### Example 10

### Preparation of (5-Methyl-2-Oxo-1,3-Dioxol-4-Yl)Methyl 2-Ethoxy-1-{[2′-(5-Oxo-4,5-Dihydro-1,2,4-Oxadiazol-3-Yl)Biphenyl-4-Yl]Methyl}-1H-Benzimidazole-7-Carboxylate (Formula II)

The (5-methyl-2-oxo-1,3-dioxol-4-yl)methyl 2-ethoxy-1-{[2′-(n′-hydroxycarbamimidoyl)biphenyl-4-yl]-methyl}-1H-benzimidazole-7-carboxylate (25 g) prepared in Example 5B and carbodiimidazole (8.96 g) were added to tetrahydrofuran (125 mL) at 20° C. to 30° C. The reaction mixture was heated to 65° C. and stirred for 4 hours. The reaction mixture was cooled to 40° C. A 5% sodium bisulphite solution (125 mL) was added to the reaction mixture and tetrahydrofuran was recovered under vacuum at 40° C. The reaction mixture was cooled to 20° C. to 30° C. and dichloromethane (175 mL) was added. The reaction mixture was washed with a 5% sodium bicarbonate solution (125 mL). Dichloromethane was completely recovered under vacuum and the solid obtained was added to acetone (75 mL) and filtered at 20° C. to 30° C. The solid material obtained was dried under vacuum to obtain the title compound.

Yield: 11.5 g

HPLC Purity: greater than 98.58%.

### Example 11

### Preparation of (5-Methyl-2-Oxo-1,3-Dioxol-4-Yl)Methyl 2-Ethoxy-1-{[2′-(5-Oxo-4,5-Dihydro-1,2,4-Oxadiazol-3 -Yl)Biphenyl-4-Yl]Methyl}-1H-Benzimidazole-7-Carboxylate (Formula II)

(5-Methyl-2-oxo-1,3-dioxol-4-yl)methyl 2-ethoxy-1-{[2′-(5-oxo-4,5-dihydro-1,2,4-oxadiazol-3-yl)biphenyl-4-yl]methyl}-1H-benzimidazole-7-carboxylate (8.5 g) was added to acetone (127.5 mL). The reaction mixture was heated to 56° C. Activated carbon (0.85 g) was added to the reaction mixture and stirred for 1 hour at 56° C. The reaction mixture was filtered through celite and concentrated to bring the volume to 90 mL. The reaction mixture was cooled to 0° C. to 5° C. and stirred for 3 to 4 hours. The solid obtained was filtered and washed with acetone (8.5 mL). The wet material was dried at 40° C. under vacuum to obtain the title compound.

Yield: 7.3 g

HPLC Purity: 99.59%

### Examples 12A and 12B

### Example 12A

### Preparation of Potassium Salt of (5-Methyl-2-Oxo-1,3-Dioxol-4-Yl)Methyl 2-Ethoxy-1-{[2′-(5-Oxo-4,5-Dihydro-1,2,4-Oxadiazol-3 -Yl)Biphenyl-4-Yl]Methyl}-1H-Benzimidazole-7-Carboxylate (Formula I)

Potassium-2-ethylhexanoate (30.44 g) was added to toluene (200 mL) at 20° C. to 30° C. under nitrogen atmosphere. The reaction mixture was heated to 100° C. to 105° C. and the toluene was azeotropically distilled to remove water and completely recovered. The reaction mixture was cooled to 40° C. to 45° C. under nitrogen and acetone (500 mL) was added to the reaction mixture. The reaction mixture was stirred and cooled to 20° C. to 30° C. The (5-methyl-2-oxo-1,3-dioxol-4-yl)methyl 2-ethoxy-1-{[2′-(5-oxo-4,5-dihydro-1,2,4-oxa diazol-3-yl)biphenyl-4-yl]methyl}-1H-benzimidazole-7-carboxylate (100 g) prepared in Example 8A was added to acetone (1300 mL) at 20° C. to 30° C. under nitrogen. The reaction mixture was cooled to −5° C. to −10° C. under nitrogen. The acetone solution of potassium-2-ethylhexanoate prepared above was added to this reaction mixture at −5° C. to −10° C. under nitrogen. The reaction mixture was stirred for 3 hours to 4 hours at −5° C. to −10° C. under nitrogen. The solid obtained was filtered at −5° C. to −10° C. under nitrogen. The reaction mixture was washed with acetone (100 mL×2) under nitrogen and dried under vacuum at 35° C. to 40° C. to obtain the title compound.

Yield: 70 g

### Example 12B

### Preparation of Potassium Salt of (5-Methyl-2-Oxo-1,3-Dioxol-4-Yl)Methyl 2-Ethoxy-1-{[2′-(5-Oxo-4,5-Dihydro-1,2,4-Oxadiazol-3-Yl)Biphenyl-4-Yl]Methyl}-1H-Benzimidazole-7-Carboxylate (Formula I)

Toluene (400 mL) and potassium-2-ethyl hexanoate (33.6 g) were heated to 110° C. and toluene was azeotropically distilled. The reaction mixture was cooled to 20° C. to 30° C. Acetone (500 mL) was added to the reaction mixture and stirred. The reaction mixture was cooled to 20° C. to 30° C. Acetone (1300 mL) was added to (5-methyl-2-oxo-1,3-dioxol-4-yl)methyl-2-ethoxy-1-{[2′-(5-oxo-4,5-dihydro-1,2,4-oxadiazol-3-yl)biphenyl-4-yl]methyl}-1H-benzimidazole-7-carboxylate (100 g) prepared in Example 9 at 20° C. to 30° C. The reaction mixture was heated to 50° C. to 55° C. The reaction mixture was filtered through celite and cooled to 0° C. to 5° C. The acetone solution of potassium-2-ethyl hexanoate prepared above was added to the reaction mixture at 0° C. to 5° C. and stirred for 3 to 4 hours. The solid obtained was filtered at 0° C. to 5° C. and washed with acetone (200 mL). The solid obtained was dried under vacuum at 20° C. to 30° C. to obtain the title compound.

Yield: 80 g

HPLC Purity: 99.9%

1. A process for the preparation of a potassium salt of azilsartan medoxomil or azilsartan medoxomil which comprises:
a) reacting a compound of Formula IV or a salt thereof, 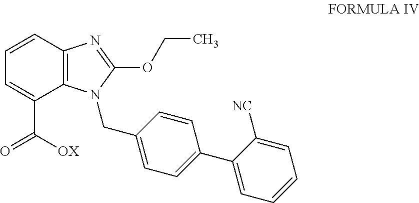
wherein X is hydrogen, a protecting group selected from alkyl, benzyl, substituted alkyl, substituted aryl with hydroxyl amine, or salts thereof, to form a compound of Formula V or a salt thereof, 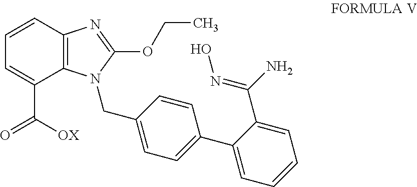
wherein X is hydrogen, a protecting group selected from alkyl, benzyl, substituted alkyl, or substituted aryl;
b) optionally hydrolyzing the protecting group of the compound of Formula V;
c) reacting the compound of Formula V with the compound of Formula VI, 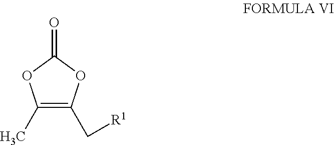
wherein R¹ is halogen or hydroxyl to form a compound of Formula VII; 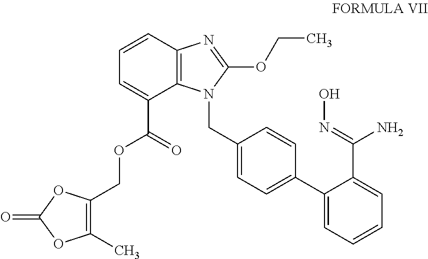 [SMILES: CCOc1nc2cccc(C(=O)OCc3oc(=O)oc3C)c2n1Cc1ccc(-c2ccccc2/C(N)=N/O)cc1]
d) optionally esterifying the compound of Formula VII to obtain a compound of Formula VIII, 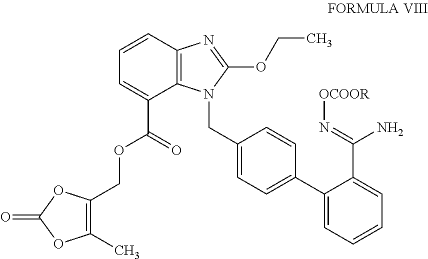
wherein R is hydrogen, alkyl, substituted alkyl, aryl, or nitro- or chloro-substituted aryl;
e) cyclizing the compound of Formula VII or Formula VIII to form azilsartan medoxomil of Formula II; 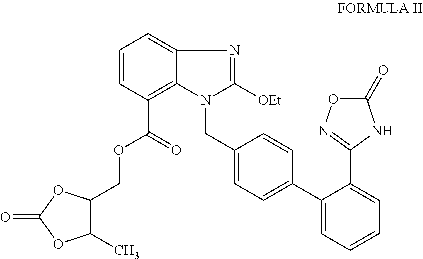 [SMILES: CCOc1nc2cccc(C(=O)OCC3OC(=O)OC3C)c2n1Cc1ccc(-c2ccccc2-c2noc(=O)[nH]2)cc1]
f) optionally isolating the azilsartan medoxomil of Formula II from the reaction mixture; and
g) optionally converting azilsartan medoxomil to a potassium salt of azilsartan medoxomil of Formula I.

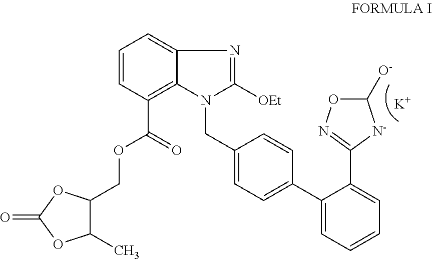 [SMILES: CCOc1nc2cccc(C(=O)OCC3OC(=O)OC3C)c2n1Cc1ccc(-c2ccccc2C2=NOC([O-])[N-]2)cc1 ; [K+]]

2. (canceled)

3. A process for the preparation of a compound of Formula VII or Formula VIII which comprises:
a) reacting a compound of Formula IV or a salt thereof, 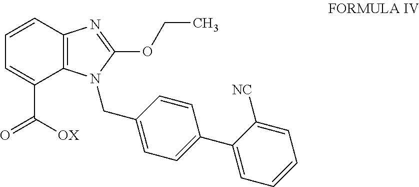
wherein X is hydrogen, a protecting group selected from alkyl, benzyl, substituted alkyl, or substituted aryl with hydroxyl amine or salts thereof to form a compound of Formula V or a salt thereof, 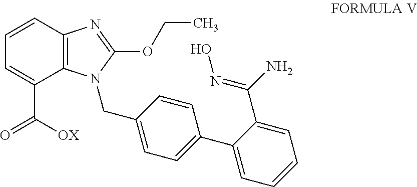
wherein X is hydrogen, a protecting group selected from alkyl, benzyl, substituted alkyl, or substituted aryl;
b) optionally hydrolyzing the protecting group of the compound of Formula V;
c) reacting the compound of Formula V with a compound of Formula VI, 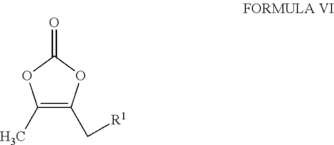
wherein R¹ is halogen or hydroxyl to form a compound of Formula VII; 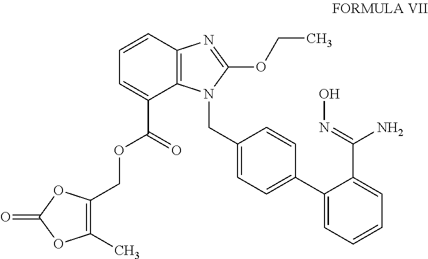 [SMILES: CCOc1nc2cccc(C(=O)OCc3oc(=O)oc3C)c2n1Cc1ccc(-c2ccccc2/C(N)=N/O)cc1]
d) optionally esterifying the compound of Formula VII to obtain a compound of Formula VIII, 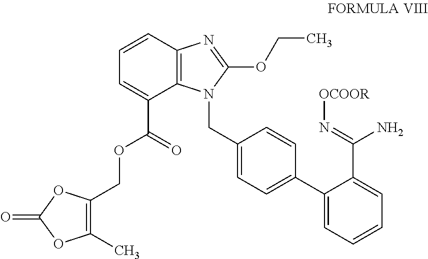
wherein R is hydrogen, alkyl, substituted alkyl, aryl, or nitro- or chloro-substituted aryl; and
e) isolating the compound of Formula VII or Formula VIII from the reaction mixture.

4. The process according to claims 1 and 3, wherein R is p-nitrophenyl.

5. The process according to claims 1 and 3, wherein the compound of Formula IV is reacted with hydroxylamine or its salt in the presence of a base and a solvent.

6. (canceled)

7. (canceled)

8. (canceled)

9. (canceled)

10. (canceled)

11. The process according to claims 1 and 3, wherein ‘X’ is methyl.

12. The process according to claims 1 and 3, wherein hydrolysis of the compound of Formula V is carried out using sodium hydroxide.

13. The process according to claims 1 and 3, wherein the compound of Formula V is reacted with the compound of Formula VI in the presence of a solvent.

14. (canceled)

15. (canceled)

16. The process according to claims 1 and 3, wherein the compound of Formula VII is esterified in the presence of a base and a solvent.

17. (canceled)

18. (canceled)

19. (canceled)

20. (canceled)

21. The process according to claims 1 and 3 wherein the esterification agent is selected from substituted or unsubstituted alkyl or aryl chloroformate.

22. (canceled)

23. The process according to claims 1 and 3, wherein the compound of Formula VII or Formula VIII is cyclized in the presence of a solvent.

24. The process according to claim 23, wherein the cyclization is thermal or chemical induced cyclization.

25. (canceled)

26. (canceled)

27. A compound of Formula VII, VIII, or IX, 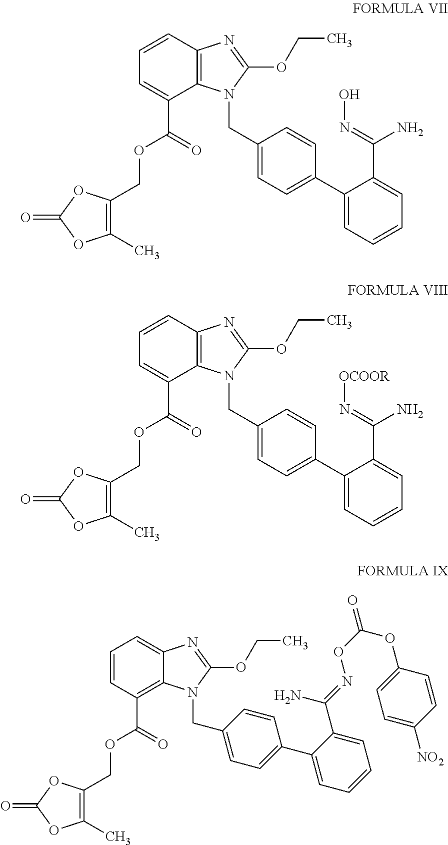
wherein R is hydrogen, alkyl, substituted alkyl, or aryl, or nitro- or chloro substituted aryl.

28. (canceled)

29. (canceled)

30. (canceled)

31. (canceled)
